(19) United States
(12) Patent Application Publication
(10) Pub. No.: US 2025/0000659 A1
(43) Pub. Date: Jan. 2, 2025

(54) HINGED ORTHOPAEDIC PROSTHESIS SYSTEM HAVING CONTROLLED CURVATURE

(71) Applicant: DePuy Ireland Unlimited Company, Ringaskiddy (IE)

(72) Inventors: Amitkumar M. Mane, South Whitley, IN (US); Lauren A. Ferris, Elkhart, IN (US); Nathan C. Reeder, Warsaw, IN (US); David E. Rottger, Auburn, IN (US)

(21) Appl. No.: 18/754,501

(22) Filed: Jun. 26, 2024

Related U.S. Application Data
(60) Provisional application No. 63/524,116, filed on Jun. 29, 2023.

(51) Int. Cl. A61F 2/38

(52) U.S. Cl. CPC A61F 2/384; A61F 2/3859; A61F 2/389

(57) ABSTRACT

A hinged orthopaedic prosthesis system includes a hinged femoral component, a housing assembly, and a tibial component. The hinged femoral component is configured to be coupled to the housing assembly such that the hinged femoral component is capable of rotation about a flexion-extension axis of rotation. The tibial component is configured to be coupled includes a tibial articular surface on which a femoral articular surface of the hinged femoral component articulates when rotated about the flexion-extension axis of rotation. Additionally, the femoral articular surface includes a curved surface section having a constant radius of curvature through a defined range of flexion of the hinged femoral component.

The present application claims priority under 35 U.S.C. §119 to, and the benefit of, U.S. Provisional Patent Application Ser. No. 63/524,116, entitled “HINGED ORTHOPAEDIC PROSTHESIS SYSTEM HAVING CONTROLLED CURVATURE,” which was filed on Jun. 29, 2023, the entirety of which is expressly incorporated herein by reference.

### TECHNICAL FIELD

The present disclosure relates to orthopaedic knee prosthesis systems and, more specifically, to hinged orthopaedic knee prostheses and methods for total knee arthroplasty procedures.

### BACKGROUND

Joint arthroplasty is a well-known surgical procedure by which a diseased and/or damaged natural joint is replaced by a prosthetic joint. A typical knee prosthesis includes a femoral component, a tibial tray, and a polymer insert or bearing positioned between the tibial tray and the femoral component. Depending on the severity of the damage to the patient's joint, orthopaedic prostheses of varying mobility may be used. For example, the knee prosthesis may include a “fixed” tibial component in some cases wherein it is desirable to limit the movement of the knee prosthesis, such as when significant soft tissue damage or loss is present. Alternatively, the knee prosthesis may include a “mobile” tibial component in cases wherein a greater degree of freedom of movement is desired. Additionally, the knee prosthesis may be a total knee prosthesis designed to replace the femoral-tibial interface of both condyles of the patient's femur or a uni-compartmental (or uni-condylar) knee prosthesis designed to replace the femoral-tibial interface of a single condyle of the patient's femur.

Movement (e.g., flexion and extension) of the natural human knee involves movement of the femur and the tibia. Specifically, during flexion and extension, the distal end of the femur and the proximal end of the tibia articulate relative to one another through a series of complex movements. Damage (e.g., trauma), disease, or revision surgeries to address those issues can deteriorate the bones, articular cartilage, and ligaments of the knee, which can ultimately affect the ability of the natural knee to function in such a manner. In such cases, orthopaedic knee prosthesis having more control over the articulation of the patent's femur and tibia may be used. One such type of knee prosthesis that may be used is a hinged knee prosthesis, which typically includes a hinge mechanism to couple the femoral component to one or both of the tibial bearing/insert and/or tibial tray components to constrain and mechanically link the components of the knee prosthesis together.

### SUMMARY

According to an aspect of the present disclosure, a hinged orthopaedic prosthesis system may include a femoral component, a housing assembly, a tibial component, and a tibial tray. The femoral component may be configured to be coupled to a surgically-prepared distal end of a patient's femur. Additionally, the femoral component may include a lateral condyle and a medial condyle spaced apart from each other and an intercondylar femoral box defined between the lateral and medial condyles. In the illustrative embodiments, at least one of the lateral and medial condyles includes a femoral articular surface.

The housing assembly may include an upper housing and a housing stem extending inferiorly from the upper housing. The upper housing may be configured to be received into the intercondylar femoral box of the femoral component to couple the femoral component to the housing assembly, and the femoral component may be rotatable relative to the housing assembly about a flexion-extension axis of rotation.

The tibial component may include a platform and a tibial stem extending downwardly from an inferior surface of the platform. The platform may include a superior surface having a tibial articular surface configured to articulate with the femoral articular surface of the femoral component. The tibial stem may include an internal passageway having an opening located on the superior surface of the platform and configured to receive the housing stem of the housing assembly.

In some embodiments, the femoral articular surface may contact the tibial articular surface at a first contact point on the femoral articular surface at a first degree of flexion of 0 degrees or less and contact the tibial articular surface at a second contact point on the femoral articular surface at a second degree of flexion greater than 90 degrees. In such embodiments, the femoral articular surface has a first curved surface section extending from the first contact point to the second contact point and having a constant radius of curvature when viewed in a sagittal plane. Additionally, an origin of the constant radius of curvature of the first curved surface section may be coincident with the flexion-extension axis of rotation.

In some embodiments, the first degree may be about 3 degrees of hyperextension. Additionally or alternatively, in some embodiments, the second degree may be greater than 100 degrees of flexion. For example, in some embodiments, the second degree is about 120 degrees of flexion. In a particular embodiment, the first degree may be about 3 degrees of hyperextension and the second degree may be about 120 degrees of flexion.

In some embodiments, the first curved surface section may include an anterior end and the femoral articular surface may further include a second curved surface section having a posterior end tangent to the anterior end of the first curved surface section and extending anteriorly from the first curved surface section. In such embodiments, the second curved surface section, when viewed in the sagittal plane, may have a first radius of curvature and a second radius of curvature different from the first radius of curvature. Each of the first and second radius of curvature may be greater than the constant radius of curvature. Additionally in some embodiments, the first curved surface section may also include a proximal end opposite the anterior end. In such embodiments, the femoral articular surface may further include a third curved surface section having a distal end tangent to the proximal end of the first curved surface section and extending proximally from the first curved surface section. The third curved surface section, when viewed in the sagittal plane, may have a third radius of curvature that is less than the first radius of curvature, the second radius of curvature, and the constant radius of curvature. Additionally, in such embodiments, the femoral articular surface may contact the tibial articular surface at a third contact point on the femoral articular surface at a third degree of flexion greater than the second degree of flexion and, when the femoral component is articulated to the third degree of flexion, a position of the flexion-extension axis of rotation relative to the tibial component may be different from a position of the flexion-extension axis of rotation when the femoral component is articulated to the second degree of flexion.

Additionally, in some embodiments, the tibial stem of the tibial component may define a tibial stem axis. In such embodiments, the flexion-extension axis of rotation may be posterior to the tibial stem axis. Additionally or alternatively, the tibial articular surface may include a dwell point that defines a distal-most point on the tibial articular surface. In such embodiments, the tibial articular surface may include a first tibial surface section posterior of the dwell point that has a constant radius of curvature. In some embodiments, the constant radius of curvature of the tibial articular surface may be equal to the constant radius of curvature of the first curved surface section. Additionally, in some embodiments, the tibial articular surface may include a second tibial surface section adjacent to and anterior of the dwell point and a third tibial surface section adjacent to and anterior to the second tibial surface section. In such embodiments, the second tibial surface section may be substantially planar and the third tibial surface section may have a constant radius of curvature.

In some embodiments, the tibial component may include a tibial insert having the platform and the tibial stem and a tibial tray, which may be separate from the tibial insert. The tibial tray may include a base plate configured to be coupled to a surgically-prepared proximal end of a patient's tibia and may have a tray stem extending downwardly from the base plate and including an internal passageway having an opening on a superior surface of the base plate and extending into the tibial tray stem, the internal passageway of the tibial tray being configured to receive the tibial stem of the tibial insert.

According to another aspect of the present disclosure, an orthopaedic prosthesis may include a hinged femoral component and a tibial component. The hinged femoral component may be configured to be coupled to a surgically-prepared distal end of a patient's femur and may include a lateral condyle and a medial condyle spaced apart from each other. Additionally, at least one of the lateral and medial condyles may include a femoral articular surface. The tibial component may include a platform having a superior surface including a tibial articular surface configured to articulate with the femoral articular surface of the femoral component.

In some embodiments, the hinged femoral component may be configured to rotate about a flexion-extension axis of rotation such that the femoral articular surface contacts the tibial articular surface at a first contact point on the femoral articular surface at a first degree of flexion of 0 degrees or less and contacts the tibial articular surface at a second contact point on the femoral articular surface at a second degree of flexion greater than 90 degrees. In such embodiments, the femoral articular surface may have a first curved surface section that extends from the first contact point to the second contact point, and the first curved surface section may have a constant radius of curvature when viewed in a sagittal plane. In some embodiments, the origin of the constant radius of curvature of the first curved surface section may be coincident with the flexion-extension axis of rotation.

Additionally, in some embodiments, the first degree may be about 3 degrees of hyperextension and the second degree may be about 120 degrees of flexion. In some embodiments, the first curved surface section may include an anterior end and a proximal end opposite the anterior end. In such embodiments, the femoral articular surface may include a second curved surface section that has a posterior end tangent to the anterior end of the first curved surface section and extends anteriorly from the first curved surface section. The second curved surface section, when viewed in the sagittal plane, may also have a first radius of curvature and a second radius of curvature different from the first radius of curvature. Additionally, each of the first and second radius of curvature may be greater than the constant radius of curvature. The femoral articular surface may further include a third curved surface section that has a distal end tangent to the proximal end of the first curved surface section and that extends proximally from the first curved surface section. The third curved surface section, when viewed in the sagittal plane, may have a third radius of curvature that is less than the first radius of curvature, the second radius of curvature, and the constant radius of curvature.

In some embodiments, the tibial component may further include a tibial stem. The tibial stem of the tibial component may define a tibial stem axis, and the flexion-extension axis of rotation may be posterior to the tibial stem axis. Additionally, in some embodiments, the tibial component may include a tibial insert having the platform and the tibial stem and a tibial tray. The tibial tray may include a base plate configured to be coupled to a surgically-prepared proximal end of a patient's tibia and may have a tray stem extending downwardly from the base plate and including an internal passageway having an opening on a superior surface of the base plate and extending into the tibial tray stem, the internal passageway of the tibial tray being configured to receive the tibial stem of the tibial insert.

Additionally, in some embodiments, the tibial articular surface may include a dwell point that defines a distal-most point on the tibial articular surface. In such embodiments, the tibial articular surface may include a first tibial surface section posterior of the dwell point that has a constant radius of curvature equal to the constant radius of curvature of the first curved surface section.

According to a further aspect of the present disclosure, an orthopaedic prosthesis system may include a primary orthopaedic prosthesis, a revision orthopaedic prosthesis, and a hinged orthopaedic prosthesis. The primary orthopaedic prosthesis may include a primary femoral component and a primary tibial component. The primary femoral component may be configured to articulate with the primary tibial component. Additionally, a distance between a medial-most point on the primary femoral component and a lateral-most point on the primary femoral component may define a medial-lateral width of the primary femoral component, and a distance between an anterior-most point on the primary femoral component and a posterior-most point on the primary femoral component may define an anterior-posterior width of the primary femoral component.

The revision orthopaedic prosthesis may include a revision femoral component and a revision tibial insert. The revision femoral component may be configured to articulate with the revision tibial component or the tibial component insert (e.g., a posterior stabilized primary tibial component). Additionally, a distance between a medial-most point on the revision femoral component and a lateral-most point on the revision femoral component may define a medial-lateral width of the revision femoral component, and a distance between an anterior-most point on the revision femoral component and a posterior-most point on the revision femoral component may define an anterior-posterior width of the revision femoral component.

The hinged orthopaedic prosthesis may include a hinged femoral component and a tibial component. The hinged femoral component may include a lateral condyle and a medial condyle spaced apart from each other. At least one of the lateral and medial condyles may include a femoral articular surface. The tibial component may include a platform that has a superior surface including a tibial articular surface configured to articulate with the femoral articular surface of the femoral component. Additionally, a distance between a medial-most point on the hinged femoral component and a lateral-most point on the hinged femoral component may define a medial-lateral width of the hinged femoral component, and a distance between an anterior-most point on the hinged femoral component and a posterior-most point on the hinged femoral component may define an anterior-posterior width of the hinged femoral component.

In some embodiments, the medial-lateral widths of the primary femoral component, the revision femoral component, and the hinged femoral component may be equal. Additionally or alternatively, the anterior-posterior widths of the primary femoral component, the revision femoral component, and the hinged femoral component may be equal. For example, the size of a condylar box of each of the primary femoral component (e.g., a posterior stabilized primary femoral component), the revision femoral component, and the hinged femoral component may be substantially the same such that no additional box cuts are required when replacing one prosthesis with another.

Additionally, in some embodiments, the femoral articular surface of the hinged femoral component may contact the tibial articular surface of the tibial component of hinged orthopaedic prosthesis at a first contact point on the femoral articular surface at about 3 degrees of hyperextension and may contact the tibial articular surface at a second contact point on the femoral articular surface at about 120 degrees of flexion. Furthermore, the femoral articular surface may have a first curved surface section extending from the first contact point to the second contact point. The first curved surface section may also have a constant radius of curvature when viewed in a sagittal plane. Additionally, in some embodiment, the origin of the constant radius of curvature of the first curved surface section may be coincident with the flexion-extension axis of rotation.

In some embodiments, the primary femoral component may include at least one primary femoral condyle that has a distal-most point and a proximal-most point on a posterior side of the primary femoral condyle. In such embodiments, a distance between the distal-most point and the proximal-most point of the primary femoral condyle may define a primary posterior condylar height of the primary femoral component. Similarly, the revision femoral component may include at least one revision femoral condyle that has a distal-most point and a proximal-most point on a posterior side of the revision femoral condyle. In such embodiments, a distance between the distal-most point and the proximal-most point of the revision femoral condyle may define a revision posterior condylar height of the revision femoral component. Furthermore, at least one of the medial condyle and lateral condyle of the hinged femoral component may include a distal-most point and a proximal-most point on a posterior side of the corresponding medial or lateral condyle. In such embodiments, a distance between the distal-most point and the proximal-most point of the corresponding medial or lateral condyle may define a hinged posterior condylar height of the hinged femoral component. In such embodiments, the hinged posterior condylar height may be greater than each of the primary posterior condylar height and the revision condylar posterior height.

### BRIEF DESCRIPTION OF THE DRAWINGS

The detailed description particularly refers to the following figures, in which:

FIG. 1 is an perspective view of an orthopaedic prosthesis system including a primary orthopaedic knee prosthesis, a revision orthopaedic knee prosthesis, and a hinged orthopaedic knee prosthesis;

FIG. 2 is an exploded perspective view of an embodiment of the hinged orthopaedic knee prosthesis of the orthopaedic prosthesis system of FIG. 1;

FIG. 3 is an anterior elevation view of an embodiment of a femoral component of the hinged orthopaedic knee prosthesis of FIG. 2;

FIG. 4 is a posterior elevation view of the femoral component of FIG. 3;

FIG. 5 is a lateral perspective view of the femoral component of FIG. 3;

FIG. 6 is a sagittal cross-sectional view of the femoral component of FIG. 3 taken generally along line 6-6 of FIG. 3;

FIG. 7 is superior plan view of the femoral component of FIG. 3;

FIG. 8 is another sagittal cross-sectional view of the femoral component of FIG. 3 taken generally along line 8-8 of FIG. 3;

FIG. 9 is a chart of illustrative lengths of radii of various sizes of the femoral component of FIG. 8;

FIG. 10 is a lateral perspective view of an embodiment of a tibial component of the hinged orthopaedic knee prosthesis of FIG. 2;

FIG. 11 is a superior plan view of the tibial component of FIG. 10;

FIG. 12 is a sagittal cross-sectional view of the tibial component of FIG. 10 taken generally along line 12-12 of FIG. 11;

FIG. 13 is another sagittal cross-sectional view of the tibial component of FIG. 10 taken generally along line 13-13 of FIG. 11; and

FIG. 14 is a sagittal cross-sectional view of the hinged orthopaedic knee prosthesis in an assembled configuration with the femoral component taken generally along the line 8-8 of FIG. 3 and the tibial component taken generally along the line 13-13 of FIG. 11.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and orthopaedic surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

The illustrative embodiments of the present disclosure are described and illustrated below to encompass prosthetic knee joints and knee joint components, as well as methods of implanting and reconstructing knee joints. It will be apparent to those of ordinary skill in the art that the preferred embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

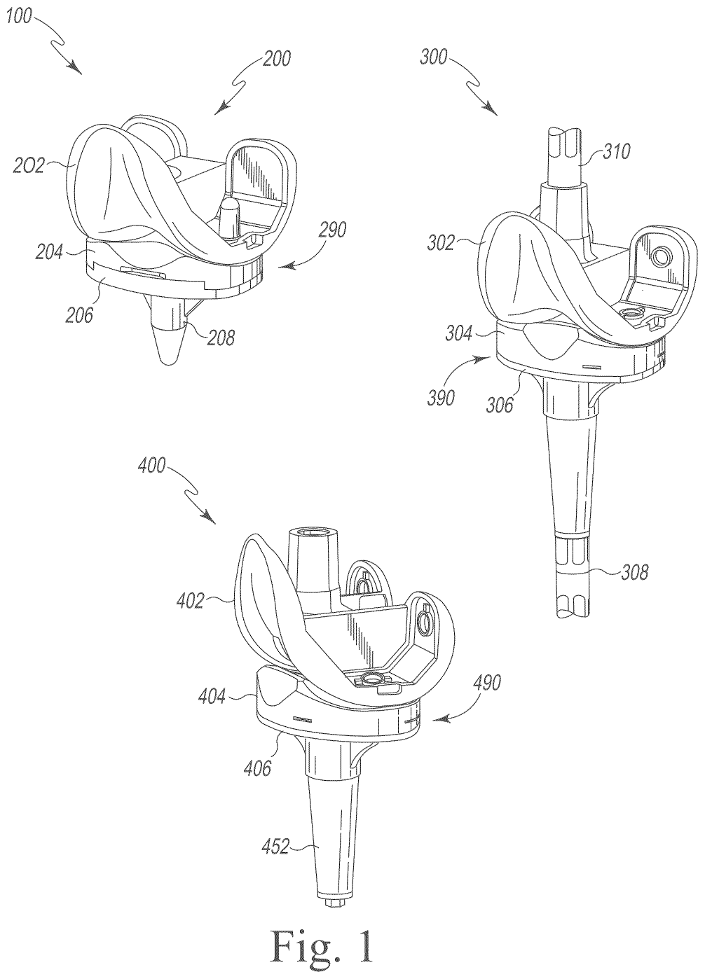

Referring now to FIG. 1, an illustrative orthopaedic prosthesis system 100 includes a primary orthopaedic prosthesis 200, a revision orthopaedic prosthesis 300, and a hinged orthopaedic prosthesis 400. Each of the prostheses 200, 300, 400 may be used by an orthopaedic surgeon to replace a patient's natural knee joint. To do so, the orthopaedic surgeon may select the appropriate prosthesis 200, 300, 400 based on a number of criteria such as the extent of trauma or disease of the patient's knee joint, the state of surrounding tissue, and/or the performance of previously implanted prostheses. For example, in some cases, the orthopaedic surgeon may initially implant the primary orthopaedic prosthesis 200 to replace the patient's joint based on an examination and indication that the patient's joint is suitable for a primary prosthesis. However, over time and under some conditions (e.g., the weakening or loss of the patient's surrounding bone and/or soft tissue), the orthopaedic surgeon may determine to replace the primary orthopaedic prosthesis 200 with the revision orthopaedic prosthesis 300, which includes additional features (e.g., a femoral and/or tibial stem) to provide additional securement of the prosthesis 300 to the patient's boney anatomy as discussed below.

Yet further, under conditions of significant bone and/or soft tissue loss or damage, the orthopaedic surgeon may determine to replace the revision orthopaedic prosthesis 300 with the hinged orthopaedic prosthesis 400, which includes further features that provide additional varus/valgus constraint and stability by physically limiting the movement of the orthopaedic knee joint as discussed below. Alternatively, in some cases, the orthopaedic surgeon may replace the primary orthopaedic prosthesis 200 with the hinged orthopaedic prosthesis 400 or implant the hinged orthopaedic prosthesis 400 as the initial orthopedic implant (e.g., in those cases in which severe disease or trauma is already present in the patient's knee joint).

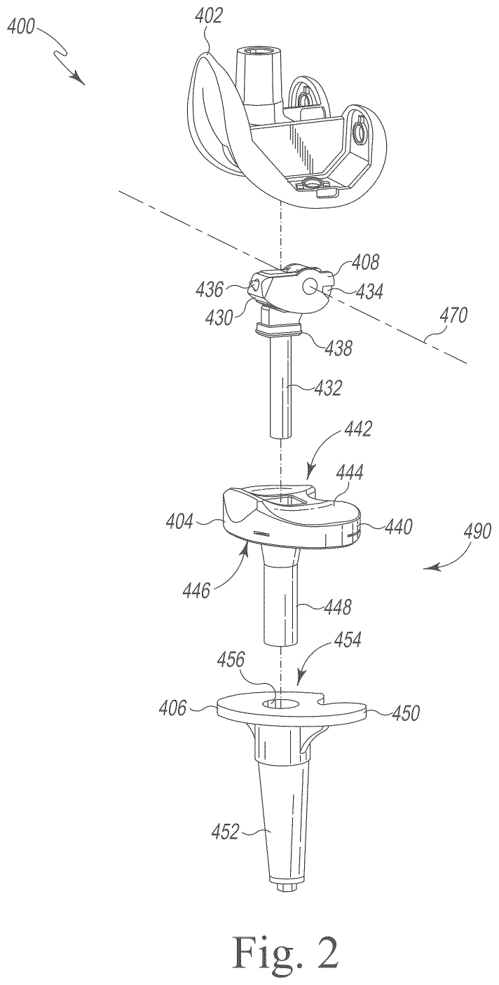

The illustrative primary orthopaedic prosthesis 200 (e.g., a posterior stabilized primary orthopaedic prosthesis) includes a primary femoral component 202 (e.g., a posterior stabilized primary femoral component), and a primary tibial component 290, which illustratively includes a primary tibial insert 204 (e.g., a posterior stabilized primary tibial insert) and a primary tibial tray 206. Similarly, the revision orthopaedic prosthesis 300 includes a revision femoral component 302 and a revision tibial component 390, which illustratively includes a revision tibial insert 304 and a revision tibial tray 306. The hinged orthopaedic prosthesis 400, however, includes a hinged femoral component 402, a housing assembly 408 (see FIG. 2), and a tibial component 490, which illustratively includes a tibial insert 404 and a tibial tray 406 (which may be identical or even the same as the revision tibial tray 306 as discussed below.) It should be appreciated that each of the tibial insert 204, 304, 404 and the corresponding tibial trays 206, 306, and 304 may be separate from each other or form a unitary tibial component 290, 390, 490, respectively. As such, as used herein, the term “tibial component” refers to embodiments in which the tibial insert and tibial tray are separate structures and to embodiments in which the tibial insert and tibial tray are combined into a single, unitary structure.

Each of the primary femoral component 202, the revision femoral component 302, and the hinged femoral component 402, as well as the primary tibial tray 206, the revision tibial tray 306, and the tibial tray 406, are illustratively formed from a metallic material such as cobalt-chromium or titanium, but may be formed from other materials, such as a ceramic material, a polymer material, a bio-engineered material, or the like, in other embodiments. Each of the primary tibial component 204, the revision tibial component 304, and the “hinged” tibial component 404 are illustratively formed from a polymer material such as an ultra-high molecular weight polyethylene (UHMWPE), but may be formed from other materials, such as a ceramic material, a metallic material, a bio-engineered material, or the like, in other embodiments.

The primary femoral component 202 is configured to be coupled to a surgically-prepared surface of the distal end of a patient's femur (not shown), and the primary tibial tray 206 is configured to be coupled to a surgically-prepared surface of the proximal end of a patient's tibia (not shown). The primary tibial tray 206 may include a tibial post 208, keels, and/or other features to facilitate the securement of the primary tibial tray 206 to the patient's tibia. The primary tibial insert 204 is configured to be coupled to the primary tibial tray 206 and engage the primary femoral component 202 to permit the primary femoral component 202 to articulate with the primary tibial insert 204 over a range of flexion. In some embodiments, the primary tibial tray 206 may include a locking mechanism or similar features to secure the primary tibial insert 204 to the primary tibial tray 206.

Similar to the primary femoral component 202, the revision femoral component 302 is configured to be coupled to a surgically-prepared surface of the distal end of the patient's femur (not shown), and the revision tibial tray 306 is configured to be coupled to a surgically-prepared surface of the proximal end of a patient's tibia (not shown). However, to provide additional support and securement of the revision orthopaedic prosthesis 300, the revision femoral component 302 may include a femoral stem 310 extending superiorly from the revision femoral component 302 and configured to be received into the femoral canal of the patient's femur. Similarly, the revision tibial tray 306 may include a tibial stem 308 extending inferiorly from the revision tibial tray 306 and configured to be received into a tibial canal of the patient's tibia. The revision tibial insert 304, similar to the primary tibial insert 204, is configured to be coupled to the revision tibial tray 306 and engage the revision femoral component 302 to permit the revision femoral component 302 to articulate with the revision tibial insert 304 over a range of flexion. In some embodiments, the revision tibial tray 306 may include a locking mechanism or similar features to secure the revision tibial insert 304 to the revision tibial tray 306, similar to the primary tibial tray 206. As such, it should be appreciated that, in some embodiments, the revision tibial insert 304 and/or the revision tibial tray 306 may be similar or identical to the primary tibial insert 204 and primary tibial tray 206, respectively. For example, in some embodiments, the primary tibial insert 204 may not be replaced during revision surgery and, in such embodiments, the revision femoral component 302 may be configured to articulate with the primary tibial insert 204 (which may be used with the revision tibial tray 306 or the “hinged” tibia tray 406).

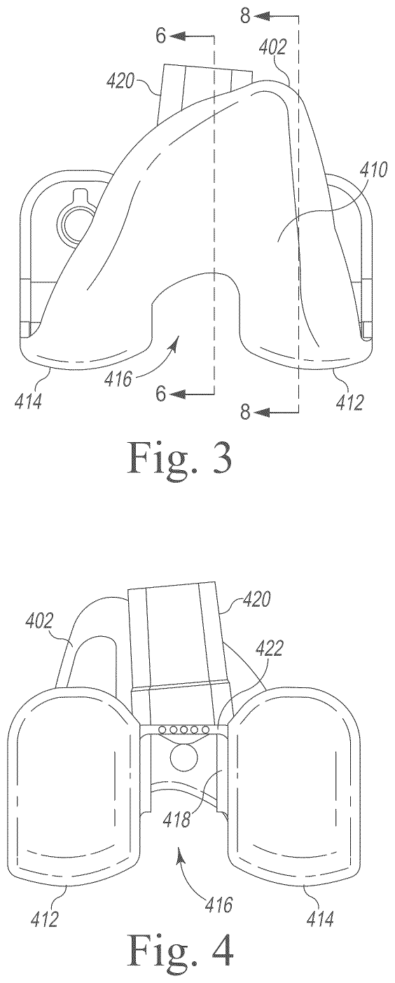

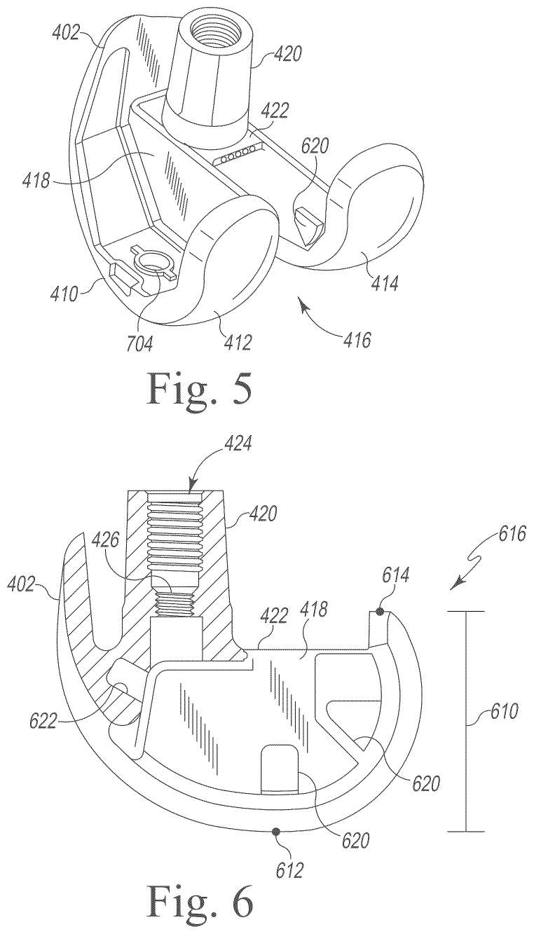

Referring now to FIG. 2-6, as discussed above, the hinged orthopaedic prosthesis 400 includes the hinged femoral component 402, the housing assembly 408, and the tibial component 490, which illustratively includes the tibial insert 404 and the tibial tray 406. As discussed in more detail below, the hinged femoral component 402 is configured to articulate with the tibial insert 404 about a flexion-extension axis of rotation 470. To do so, as best shown in FIGS. 3-5, the hinged femoral component 402 includes an outer, femoral articular surface 410 having a lateral condyle 412 and a medial condyle 414, which are configured to articulate on a corresponding tibial articular surface 444 of the tibial insert 404 as discussed in more detail below.

The lateral and medial condyles 412, 414 are spaced apart to define an intercondylar notch or opening 416 therebetween. An intercondylar femoral box 418 is located within the intercondylar opening 416 and includes a femoral post 420 extending superiorly from a superior side 422 of the intercondylar femoral box 418. As best shown in FIG. 6, the illustrative femoral post 420 includes a threaded internal passageway 424, which facilitates the attachment of an optional femoral stem (not shown) to the hinged femoral component 402. Additionally, the femoral post 420 may include a distal threaded aperture 426, which generally has a smaller cross-section than the threaded internal passageway 424 and is configured for use with an associated removal rod or similar tool to facilitate the removal of the hinged femoral component 402.

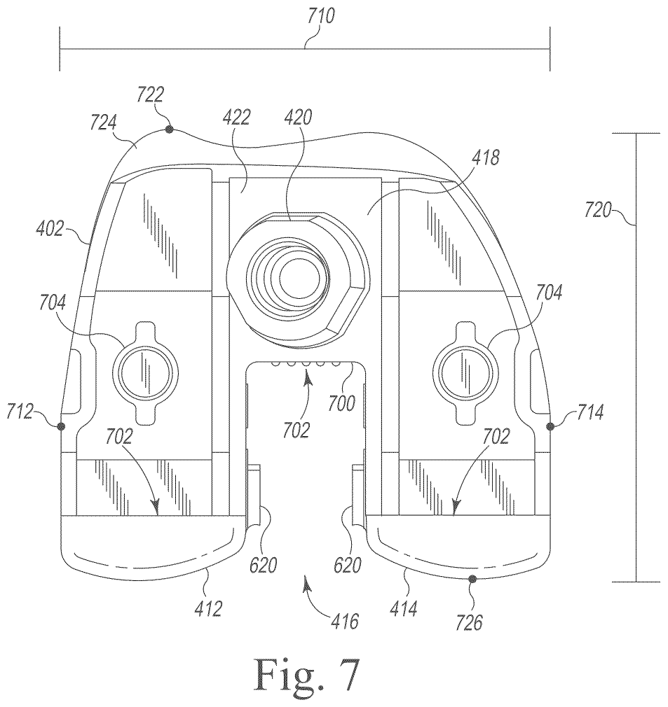

Referring now to FIG. 7, in some embodiments, the hinged femoral component 402 is shaped and sized to facilitate the use of revision accessories with the hinged femoral component 402. For example, in the illustrative embodiment, the femoral post 420 of the hinged femoral component 402 may be shaped, sized, and positioned on the intercondylar femoral box 418 (e.g., in the same anterior-posterior and medial-lateral position) such that femoral post 420 is usable with a revision stem, sleeve, offset adaptor, and/or other accessories that are configured to be attached to the revision femoral component 302. Additionally, the hinged femoral component 402 includes alignment features 702 located on a proximal sidewall 700 of the intercondylar femoral box 418 that allow the use of a revision sleeve (i.e., a sleeve configured for use with the revision femoral component 302) at various degrees of rotation (e.g., at 20 degrees of internal rotation, 10 degrees of internal rotation, neutral rotation, 10 degrees of external rotation, 20 degrees of external rotation, etc.). Furthermore, the hinged femoral component 402 may include augment mounts 704 sized and located on the proximal side of the hinged femoral component 402 to facilitate the use of medial, lateral, distal, and posterior augments (not shown). As such, it should be appreciated that the hinged femoral component 402 may be used with various revision accessories while providing additional varus/valgus constraint, relative to the revision femoral component 302, due to the hinged fixation of the femoral component 402.

Additionally, in some embodiments, the hinged femoral component 402 is sized so as to have a similar or identical “envelope” size as the primary and revision femoral components 202, 302. For example, the hinged femoral component 402 has a medial-lateral width 710 and an anterior-posterior width 720 that is identical to, or substantially similar to (e.g., equal to within a manufacturing tolerance), a corresponding medial-lateral width and anterior-posterior width of the primary femoral component 202 and/or the revision femoral component 302. The medial-lateral width 710 of the hinged femoral component 402 (as well as the primary and revision femoral components 202, 302) is defined as the distance between a medial-most point 714 located on the medial condyle 414 and a lateral-most point 712 located on the lateral condyle 412. Similarly, the anterior-posterior width 720 of the hinged femoral component 402 (as well as the primary and revision femoral components 202, 302) is defined as the distance between an anterior-most point 722 located on an anterior flange 724 of the outer, articular surface 410 and a posterior-most point 726 located on the lateral and/or medial condyle 412, 414.

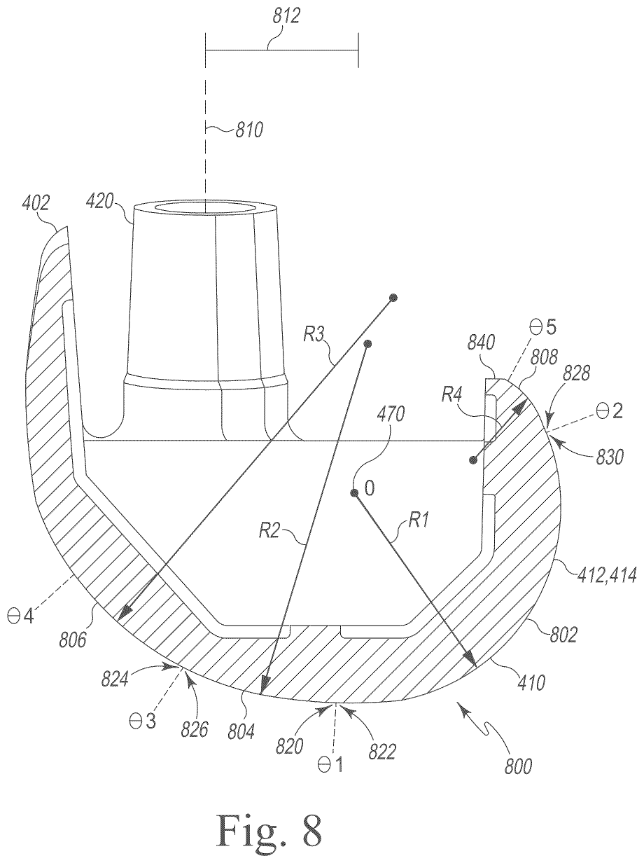

However, to facilitate the condylar curvature of the lateral and medial condyles 412, 414, which is discussed in more detail below in regard to FIG. 8, the hinged femoral component 402 has a posterior condylar height 610 (see FIG. 6) that is greater than the posterior condylar height of each of the primary and revision femoral components 202, 302. The posterior condylar height 610 of the hinged femoral component 402 (as well as the primary and revision femoral components 202, 302) is defined as the distance between a distal-most point 612 on at least one of the lateral or medial condyles 412, 414 and a proximal-most point 614 on a posterior side 616 of the corresponding lateral or medial condyles 412, 414.

Referring now to FIG. 8, the lateral and medial condyles 412, 414 of the femoral articular surface 410 of the hinged femoral component 402 are configured to articulate on the tibial articular surface 444 of the tibial insert 404 (see FIG. 2) as discussed above. As such, each of the lateral and medial condyles 412, 414 includes a condyle surface 800 having a condyle curvature shaped to facilitate the hinged rotation of the femoral component 402 on the tibial insert 404. The condyle surface 800 is convexly curved in the sagittal plane as shown in FIG. 8 and is formed from a number of curved surface sections 802, 804, 806, and 808, each of which is tangent to the adjacent curved surface section(s). Each curved surface section 802, 804, 806, and 808 contacts the tibial insert 404 through a different range of degrees of flexion. For example, the curved surface section 802 contacts the tibial insert 404 during a range of early-to-late flexion, the curved surface sections 804, 806 contact the tibial insert 404 during a range of hyperextension, and the curved surface section 808 contacts the tibial insert 404 during a range of hyperflexion.

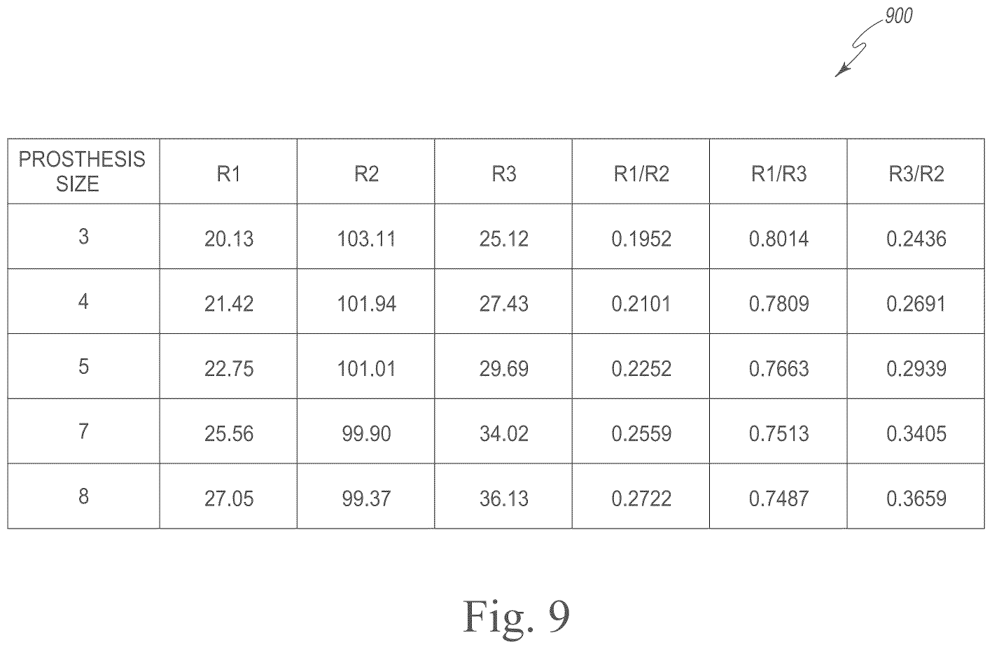

Each curved surface section 802, 804, 806, and 808 is defined by a constant radius of curvature R1, R2, R3, and R4, respectively. In the illustrative embodiment, the radius of curvature R2 of the curved surface section 804 and the radius of curvature R3 of the curved surface section 806 are both greater than the radius of curvature R1 of the curved surface section 802. Additionally, each of the radius of curvature R1, R2, and R3 is greater than the radius of curvature R4 of the curved surface section 808. For example, FIG. 9 illustrates a table of illustrative lengths of the constant radius of curvatures R1, R2, and R3 across a selection of size. As shown in FIG. 9, the constant radius of curvature R1 has a length in the range of about 20.00 millimeters to about 27.25 millimeters, the constant radius of curvature R2 has a length in the range of about 103.25 millimeters to about 99.50 millimeters, and the constant radius of curvature R3 has a length in the range of about 25.00 millimeters to about 36.25 millimeters. Additionally, to support the additional varus/valgus stability provided by the hinged orthopaedic prosthesis 400, the ratios of R1/R2, R1/R3, and R2/R3 fall within a defined range. For example, the ratio of R1/R2 has a value in the range of about 0.19 to about 0.28 and, in the illustrative embodiment, in the range of about 0.1952 to about 0.2722. The ratio of R1/R3 has a value in the range of about 0.74 to about 0.81 and, in the illustrative embodiment, in the range of about 0.7487 to about 0.8014. Similarly, the ratio of R3/R2 has a value in the range of about 0.24 to about 0.37 and, in the illustrative embodiment, in the range of about 0.2436 to about 0.3659. It should be appreciated that the lengths and ratios provided above are for the illustrative size range of size 3 to size 8 and that the provided lengths and ratios may vary if additional or different sizes are considered.

Referring back to FIG. 8, in the illustrative embodiment, the curved surface section 802 defined by the radius of curvature R1 is configured to contact the tibial insert 404 during a typical use range of flexion and extends from a first degree of flexion θ1 to a second degree of flexion θ2. The particular range of flexion of the curved surface section 802 may be based on the various criteria including the desired movement characteristics of the hinged femoral component 402, the size of the hinged femoral component 402, aspects of the individual patient, and/or other factors. For example, in some embodiments, the first degree of flexion θ1 may be zero degrees of flexion or less (i.e., hyperextension). Additionally, in some embodiments, the second degree of flexion θ2 may be at least 90 degrees of flexion, at least 100 degrees of flexion, at least 120 degrees of flexion, or greater. In one particular embodiment, the first degree of flexion θ1 is equal to or about 3 degrees of hyperextension and the second degree of flexion θ2 is equal to or about 120 degrees of flexion.

As shown in FIG. 8, the origin O of the radius of curvature R1 that defines the curved surface section 802 is coincident with, or otherwise lies on, the flexion-extension axis of rotation 470 of the hinged femoral component 402 (see, also, FIG. 2). As such, due the positioning of the origin O and the constant radius of curvature R1, the inferior-superior position of the hinged femoral component 402 relative to the tibial insert 404 remains substantially constant during the normal range of flexion (e.g., a range of flexion between the first degree of flexion θ1 and the second degree of flexion θ2). In this way, “pistoning” of the femoral component 402 relative to the tibial insert 404 (i.e., the vertical movement of the femoral component 402) is reduced or otherwise eliminated. Additionally, as shown in FIG. 8, the origin O of the radius of curvature R1 of the curved surface section 802 lies posterior of an axis 810 defined by the femoral post 420 by a distance 812.

The curved surface section 804 defined by the radius of curvature R2 is configured to contact the tibial insert 404 during a range of hyperextension and extends from the first degree of flexion θ1 to a third degree of flexion θ3, which is less than the first degree of flexion θ1. That is, the third degree of flexion θ3 is a greater angle of hyperextension than the first degree of flexion θ1. As discussed above, the curved surface section 804 is tangent to the curved surface section 802 and includes a posterior end 820 tangent to an anterior end 822 of the curved surface section 802.

Similar to the curved surface section 804, the curved surface section 806 defined by the radius of curvature R3 is configured to contact the tibial insert 404 during a range of hyperextension and extends from the third degree of flexion θ3 to a fourth degree of flexion θ4, which is less than the third degree of flexion θ3. That is, the fourth degree of flexion θ3 is a greater angle of hyperextension than the third degree of flexion θ3. The curved surface section 806 is tangent to the curved surface section 804 and includes a posterior end 824 tangent to an anterior end 826 of the curved surface section 804.

The curved surface section 808 defined by the radius of curvature R4 is configured to contact the tibial insert 404 during a range of hyperflexion and extends from the second degree of flexion θ2 to a fifth degree of flexion θ5, which is greater than the second degree of flexion θ2. As discussed above, the curved surface section 808 is tangent to the curved surface section 802, and the curved surface section 808 includes a distal end 828 tangent to a proximal end 830 of the curved surface section 802. The curved surface section 808 is designed so as to wrap the sagittal curvature of the condyle surface 800 from the curved surface section 802 to a flat or planar proximal section 840 of the condyle surface 800.

Referring back to FIG. 2, the illustrative housing assembly 408 includes an upper housing 430 and a housing stem 432 extending inferiorly from the upper housing 430. The housing assembly 408 is configured to be attached to the hinged femoral component 402. For example, the upper housing 430 is shaped and sized to be received into the intercondylar femoral box 418. The upper housing 430 includes attachment features 434 configured to cooperate with or otherwise mate with corresponding attachment features 620 of the hinged femoral component 402 (see FIGS. 6 & 7) to thereby attach the hinged femoral component 402 to the housing assembly 408.

Additionally, in the illustrative embodiment, the housing assembly 408 includes a cross pin 436 extending outwardly from the upper housing 430. The cross pin 436 is configured to be received in a corresponding cross pin aperture 622 (see FIG. 6) defined in the intercondylar femoral box 418 of the hinged femoral component 402 to secure the hinged femoral component 402 to the housing assembly 408. When so secured, the hinged femoral component 402 is capable of rotating, relative to the upper housing 430, about the axis of rotation 470 as discussed above.

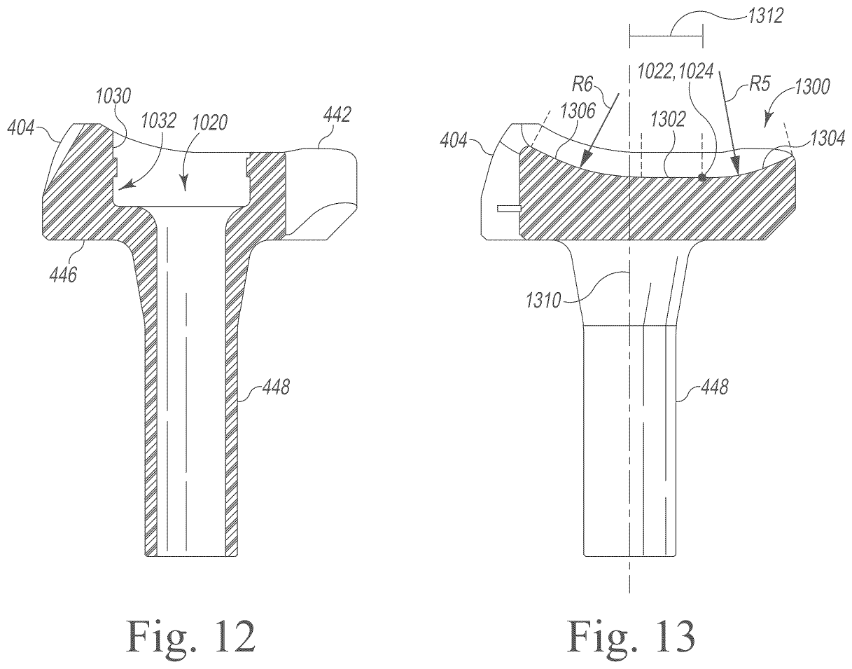

The Illustrative housing assembly 408 also includes a mounting flange 438 located toward the inferior side of the upper housing 430 and extending outwardly from the housing stem 432. The mounting flange 438 is configured to be received into a corresponding mounting undercut or recess 1032 of an internal passageway 1020 of the tibial insert 404 (see FIG. 12) to secure the housing assembly 408 to the tibial insert 404. Illustratively, the mounting flange 438 has a rectangular shape and is configured to restrict or otherwise limit rotational movement of the housing assembly 408 relative to the tibial insert 404 while the housing assembly 408 is coupled to the tibial insert 404. It should be appreciated, however, that the mounting flange 438 may have other geometric shapes in other embodiments.

The tibial insert 404 of the hinged orthopaedic prosthesis 400 includes a platform 440 having a superior surface 442, which includes the tibial articular surface 444, and an inferior surface 446 opposite the superior surface 442. Additionally, the tibial insert 404 includes a tibial stem 448 extending inferiorly from the inferior surface 446 of the platform 440.

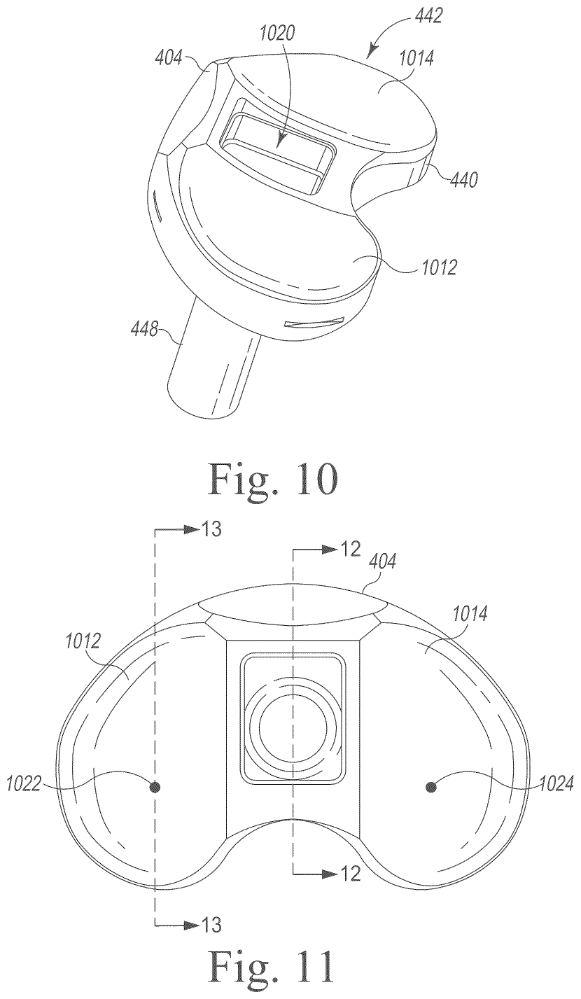

As discussed above, the tibial articular surface 444 is configured to articular with the femoral articular surface 410 of the hinged femoral component 402. To do so, as shown in FIGS. 10-13, the tibial articular surface 444 includes a lateral articular surface 1012 configured to articulate with the lateral condyle 412 of the hinged femoral component 402 and a medial articular surface 1014 configured to articulate with the medial condyle 414 of the hinged femoral component 402. As discussed above, the tibial insert 404 also includes the elongated, internal passageway 1020 (see FIG. 12), which extends longitudinally through the tibial stem 448. The internal passageway 1020 includes an opening 1030 located on the superior surface 442 of the platform 440 between the lateral articular surface 1012 and the medial articular surface 1014. The internal passageway 1020 and associated opening 1030 are sized and configured to receive the housing stem 432 of the housing assembly 408 to facilitate the coupling of the housing assembly 408 and femoral component 402 to the tibial insert 404. For example, the internal passageway 1020 has a diameter and length greater than the diameter and length of the housing stem 432, which allows some amount of “pistoning” of the housing stem 432 under certain conditions. Additionally, as discussed above with regard to FIG. 2, the internal passageway 1020 of the tibial insert 404 includes the mounting undercut 1032 configured to receive the mounting flange 438 of the housing assembly 408 to facilitate the attachment of the housing assembly 408 to the tibial insert 404.

As shown in FIG. 11, each of the lateral and medial articular surfaces 1012, 1014 includes a corresponding dwell point 1022, 1024, respectively. The dwell point 1022 defines the distal-most point of the lateral articular surface 1012, and the dwell point 1024 defines the distal-most point of the medial articular surface 1014. As shown in FIG. 13, each of the dwell points 1022, 1024 lie posteriorly of an axis 1310 defined by the tibial stem 448 by a distance 1312.

As discussed above, the lateral articular surface 1012 is configured to articulate with the lateral condyle 412 of the hinged femoral component 402 and the medial articular surface 1014 is configured to articulate with the medial condyle 414 of the hinged femoral component 402. As such, each of the lateral and medial articular surfaces 1012, 1014 includes an articular surface 1300 having a curvature shaped to facilitate the hinged (flexion-extension) rotation of the femoral component 402 on the tibial insert 404. The articular surface 1300 is generally concavely curved in the sagittal plane as shown in FIG. 13 and is formed from a number of surface sections 1302, 1304, 1306. The surface section 1302 is semi-planar and extends anteriorly from the corresponding dwell point 1022, 1024. As used herein, the term “semi-planar” refers to a surface section that is either planar or is otherwise defined by a radius of curvature that is at least three times the length of the radius of curvature of an adjacent curved section. It should be appreciated that due to the semi-planar nature of the anterior surface section 1302 and its adjacency to the dwell point 1022, 1024, the anterior surface section 1302 may form a “dwell region” in some embodiments.

The surface section 1304 is curved in the sagittal plane and extends posteriorly of the corresponding dwell point 1022, 1024. The surface section 1304 is defined by a constant radius of curvature R5. In the illustrative embodiment, the constant radius of curvature R5 is equal to, or otherwise substantially similar to, the constant radius of curvature R1 that defines the femoral curved surface section 802. Similarly, the surface section 1306 is curved in the sagittal plane and extends anteriorly of the corresponding dwell point 1022, 1024. The surface section 1306 is defined by a constant radius of curvature R6. In the illustrative embodiment, the value of the constant radius of curvature R6 is different from value of the constant radius of curvature R5.

Referring now back to FIG. 2, the tibial tray 406 includes a base plate 450 configured to be coupled to a surgically-prepared proximal end of the patient's tibia (not shown). As discussed above, the tibial tray 406 may be identical to the revision tibial tray 306. For example, in some embodiments, the hinged femoral component 402, the housing assembly 408, and the tibial insert 404 may be configured to be used with the revision tibial tray 306 such that the tibial tray 306 need not be replaced during the orthopaedic surgical procedure for implanting the hinged orthopaedic prosthesis 400.

The illustrative tibial tray 406 includes a tibial tray stem 452 that extends inferiorly or downwardly from the base plate 450. The tibial tray stem 452 includes an internal passageway 454 that extends longitudinally through the tibial tray stem 452. The internal passageway 454 includes an opening 456 located on the base plate 450. The internal passageway 454 and associated opening 456 are shaped, sized, and configured to receive the tibial stem 448 of the tibial insert 404 to facilitate the coupling of the tibial insert (and the femoral component 402 and housing assembly 408) to the tibial tray 406.

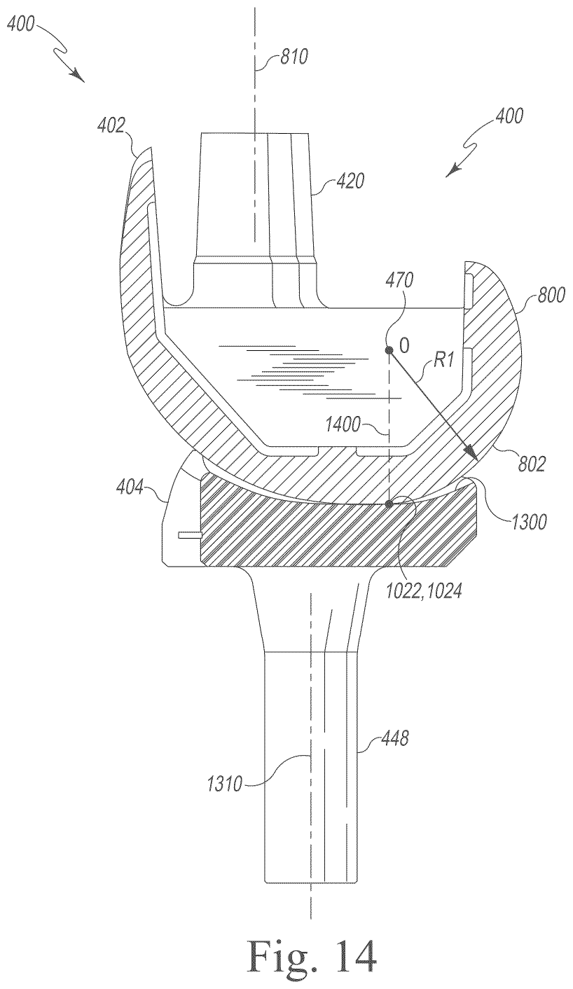

Referring now to FIG. 14, in use and as discussed above, the hinged femoral component 402 is configured to articulate on the tibial insert 404 through a range of flexion. As shown in FIG. 14, when the femoral component 402 is coupled to the tibial insert 404, the origin O of the radius of curvature R1 and the dwell points 1022, 1024 of the tibial articular surface 1300 are located posteriorly of the axis 810 defined by the femoral post 420 and the axis 1310 defined by the tibial stem 448. Additionally, at full extension as shown in FIG. 14, the origin O of the constant radius of curvature R1 that defines the curved surface section 802 of the condyle surface 800 of the hinged femoral component 402 is located directly superior of the dwell point 1022, 1024 of the corresponding lateral/medial articular surface 1012, 1014 of the tibial articular surface 1300 of the tibial insert 404 as indicated by line 1400. Due to the constant radius of curvature R1 and the hinged arrangement of the femoral component 402, the origin O remains in that position throughout the range of operational flexion (e.g., from the first degree of flexion θ1 to the second degree of flexion θ2). In this way, the hinged orthopaedic prosthesis 400 provides an amount of varus/valgus constraint and stability throughout the range of operational flexion.

It should be appreciated that, in some embodiments, the primary orthopaedic prosthesis 200, the revision orthopaedic prosthesis 300, and the hinged orthopaedic prosthesis 400 form a system of orthopaedic prostheses configured to improve the ease of replacement of primary orthopaedic prosthesis 200 with the revision orthopaedic prosthesis 300 and the revision orthopaedic prosthesis 300 with the hinged orthopaedic prosthesis 400. For example, in some embodiments, the size of the condylar box of each of the prostheses 200, 300, and 400 are substantially the same size such that no additional box cuts are required when replacing one prosthesis with another (e.g., replacing the revision orthopaedic prosthesis 300 with the hinged orthopaedic prosthesis 400.) Additionally, the prostheses 200, 300, and 400 may have common features across each prosthesis to facilitate similar use. For example, in some embodiments, the trochlear grove of each prosthesis 200, 300, and 400 has a similar or identical geometry to support similar tracking of the natural or prosthetic patella across the different the prostheses 200, 300, and 400.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A hinged orthopaedic prosthesis system, comprising:
a femoral component configured to be coupled to a surgically-prepared distal end of a patient's femur, the femoral component including a lateral condyle and a medial condyle spaced apart from each other and an intercondylar femoral box defined between the lateral and medial condyles, wherein at least one of the lateral and medial condyles includes a femoral articular surface;
a housing assembly including an upper housing and a housing stem extending inferiorly from the upper housing, wherein the upper housing is configured to be received into the intercondylar femoral box of the femoral component to couple the femoral component to the housing assembly, wherein the femoral component is rotatable relative to the housing assembly about a flexion-extension axis of rotation; and
a tibial component including (i) a platform having a superior surface including a tibial articular surface configured to articulate with the femoral articular surface of the femoral component and (ii) a tibial stem extending downwardly from an inferior surface of the platform and including an internal passageway having an opening located on the superior surface of the platform and configured to receive the housing stem of the housing assembly;
wherein the femoral articular surface contacts the tibial articular surface at a first contact point on the femoral articular surface at a first degree of flexion of 0 degrees or less and contacts the tibial articular surface at a second contact point on the femoral articular surface at a second degree of flexion greater than 90 degrees,
wherein the femoral articular surface has a first curved surface section extending from the first contact point to the second contact point and wherein the first curved surface section has a constant radius of curvature when viewed in a sagittal plane and an origin of the constant radius of curvature of the first curved surface section is coincident with the flexion-extension axis of rotation.

2. The hinged orthopaedic prosthesis system of claim 1, wherein the first degree is about 3 degrees of hyperextension.

3. The hinged orthopaedic prosthesis system of claim 1, wherein the second degree is greater than 100 degrees of flexion.

4. The hinged orthopaedic prosthesis system of claim 3, wherein the second degree is about 120 degrees of flexion.

5. The hinged orthopaedic prosthesis system of claim 1, wherein the first degree is about 3 degrees of hyperextension and the second degree is about 120 degrees of flexion.

6. The hinged orthopaedic prosthesis system of claim 1, wherein the first curved surface section includes an anterior end, and
wherein the femoral articular surface further includes a second curved surface section having a posterior end tangent to the anterior end of the first curved surface section and extending anteriorly from the first curved surface section, wherein the second curved surface section, when viewed in the sagittal plane, has a first radius of curvature and a second radius of curvature different from the first radius of curvature, and wherein each of the first and second radius of curvature is greater than the constant radius of curvature.

7. The hinged orthopaedic prosthesis system of claim 6, wherein the first curved surface section includes a proximal end opposite the anterior end, and
wherein the femoral articular surface further includes a third curved surface section having a distal end tangent to the proximal end of the first curved surface section and extending proximally from the first curved surface section, wherein the third curved surface section, when viewed in the sagittal plane, has a third radius of curvature that is less than the first radius of curvature, the second radius of curvature, and the constant radius of curvature,
wherein the femoral articular surface contacts the tibial articular surface at a third contact point on the femoral articular surface at a third degree of flexion greater than the second degree of flexion and wherein when the femoral component is articulated to the third degree of flexion, a position of the flexion-extension axis of rotation relative to the tibial component is different from a position of the flexion-extension axis of rotation when the femoral component is articulated to the second degree of flexion.

8. The hinged orthopaedic prosthesis system of claim 1, wherein the tibial stem of the tibial component defines a tibial stem axis and wherein the flexion-extension axis of rotation is posterior to the tibial stem axis.

9. The hinged orthopaedic prosthesis system of claim 1, wherein the tibial articular surface includes a dwell point that defines a distal-most point on the tibial articular surface and wherein the tibial articular surface includes a first tibial surface section posterior of the dwell point that has a constant radius of curvature.

10. The hinged orthopaedic prosthesis system of claim 9, wherein the constant radius of curvature of the tibial articular surface is equal to the constant radius of curvature of the first curved surface section.

11. The hinged orthopaedic prosthesis system of claim 9, wherein the tibial articular surface includes a second tibial surface section adjacent to and anterior of the dwell point and a third tibial surface section adjacent to and anterior to the second tibial surface section,
wherein the second tibial surface section is substantially planar and the third tibial surface section has a constant radius of curvature.

12. The hinged orthopaedic prosthesis system of claim 1, wherein the tibial component includes (i) a tibial insert having the platform and the tibial stem and (ii) a tibial tray having a base plate configured to be coupled to a surgically-prepared proximal end of a patient's tibia and having a tray stem extending downwardly from the base plate and including an internal passageway having an opening on a superior surface of the base plate and extending into the tibial tray stem, the internal passageway of the tibial tray being configured to receive the tibial stem of the tibial insert.

13. An orthopaedic prosthesis, comprising:
a hinged femoral component configured to be coupled to a surgically-prepared distal end of a patient's femur, the hinged femoral component including a lateral condyle and a medial condyle spaced apart from each other, wherein at least one of the lateral and medial condyles includes a femoral articular surface; and
a tibial component including a platform having a superior surface including a tibial articular surface configured to articulate with the femoral articular surface of the femoral component,
wherein the hinged femoral component is configured to rotate about a flexion-extension axis of rotation such that the femoral articular surface contacts the tibial articular surface at a first contact point on the femoral articular surface at a first degree of flexion of 0 degrees or less and contacts the tibial articular surface at a second contact point on the femoral articular surface at a second degree of flexion greater than 90 degrees,
wherein the femoral articular surface has a first curved surface section extending from the first contact point to the second contact point and wherein the first curved surface section has a constant radius of curvature when viewed in a sagittal plane and an origin of the constant radius of curvature of the first curved surface section is coincident with the flexion-extension axis of rotation.

14. The orthopaedic prosthesis of claim 13, wherein the first degree is about 3 degrees of hyperextension and the second degree is about 120 degrees of flexion.

15. The orthopaedic prosthesis of claim 13, wherein the first curved surface section includes an anterior end and a proximal end opposite the anterior end,
wherein the femoral articular surface includes a second curved surface section having a posterior end tangent to the anterior end of the first curved surface section and extending anteriorly from the first curved surface section, wherein the second curved surface section, when viewed in the sagittal plane, has a first radius of curvature and a second radius of curvature different from the first radius of curvature, and wherein each of the first and second radius of curvature is greater than the constant radius of curvature, and
wherein the femoral articular surface further includes a third curved surface section having a distal end tangent to the proximal end of the first curved surface section and extending proximally from the first curved surface section, wherein the third curved surface section, when viewed in the sagittal plane, has a third radius of curvature that is less than the first radius of curvature, the second radius of curvature, and the constant radius of curvature.

16. The orthopaedic prosthesis of claim 13, wherein the tibial component further includes a tibial stem that defines a tibial stem axis and wherein the flexion-extension axis of rotation is posterior to the tibial stem axis.

17. The orthopaedic prosthesis of claim 16, wherein the tibial component includes (i) a tibial insert having the platform and the tibial stem and (ii) a tibial tray having a base plate configured to be coupled to a surgically-prepared proximal end of a patient's tibia and having a tray stem extending downwardly from the base plate and including an internal passageway having an opening on a superior surface of the base plate and extending into the tibial tray stem, the internal passageway of the tibial tray being configured to receive the tibial stem of the tibial insert.

18. The orthopaedic prosthesis of claim 13, wherein the tibial articular surface includes a dwell point that defines a distal-most point on the tibial articular surface and wherein the tibial articular surface includes a first tibial surface section posterior of the dwell point that has a constant radius of curvature equal to the constant radius of curvature of the first curved surface section.

19. An orthopaedic prosthesis system, comprising:
a primary orthopaedic prosthesis including a primary femoral component and a primary tibial component, wherein the primary femoral component is configured to articulate with the primary tibial component, and wherein a distance between a medial-most point on the primary femoral component and a lateral-most point on the primary femoral component defines a medial-lateral width of the primary femoral component and a distance between an anterior-most point on the primary femoral component and a posterior-most point on the primary femoral component defines an anterior-posterior width of the primary femoral component;
a revision orthopaedic prosthesis including a revision femoral component and a revision tibial component, wherein the revision femoral component is configured to articulate with the revision tibial component, and wherein a distance between a medial-most point on the revision femoral component and a lateral-most point on the revision femoral component defines a medial-lateral width of the revision femoral component and a distance between an anterior-most point on the revision femoral component and a posterior-most point on the revision femoral component defines an anterior-posterior width of the revision femoral component; and
a hinged orthopaedic prosthesis including (i) a hinged femoral component including a lateral condyle and a medial condyle spaced apart from each other, wherein at least one of the lateral and medial condyles includes a femoral articular surface, and (ii) a tibial component including a platform having a superior surface including a tibial articular surface configured to articulate with the femoral articular surface of the femoral component, wherein a distance between a medial- most point on the hinged femoral component and a lateral-most point on the hinged femoral component defines a medial-lateral width of the hinged femoral component and a distance between an anterior-most point on the hinged femoral component and a posterior-most point on the hinged femoral component defines an anterior-posterior width of the hinged femoral component;
wherein the medial-lateral widths of the primary femoral component, the revision femoral component, and the hinged femoral component are equal and wherein the anterior-posterior widths of the primary femoral component, the revision femoral component, and the hinged femoral component are equal,
wherein the femoral articular surface of the hinged femoral component contacts the tibial articular surface of the tibial component of hinged orthopaedic prosthesis at a first contact point on the femoral articular surface at about 3 degrees of hyperextension and contacts the tibial articular surface at a second contact point on the femoral articular surface at about 120 degrees of flexion,
wherein the femoral articular surface has a first curved surface section extending from the first contact point to the second contact point and wherein the first curved surface section has a constant radius of curvature when viewed in a sagittal plane and an origin of the constant radius of curvature of the first curved surface section is coincident with the flexion-extension axis of rotation.

20. The orthopaedic prosthesis system of claim 19, wherein:
the primary femoral component includes at least one primary femoral condyle having a distal-most point and a proximal-most point on a posterior side of the primary femoral condyle, wherein a distance between the distal-most point and the proximal-most point of the primary femoral condyle defines a primary posterior condylar height of the primary femoral component,
the revision femoral component includes at least one revision femoral condyle having a distal-most point and a proximal-most point on a posterior side of the revision femoral condyle, wherein a distance between the distal-most point and the proximal-most point of the revision femoral condyle defines a revision posterior condylar height of the revision femoral component,
at least one of the medial condyle and lateral condyle of the hinged femoral component includes a distal-most point and a proximal-most point on a posterior side of the corresponding medial or lateral condyle, wherein a distance between the distal-most point and the proximal-most point of the corresponding medial or lateral condyle defines a hinged posterior condylar height of the hinged femoral component, and
wherein the hinged posterior condylar height is greater than each of the primary posterior condylar height and the revision posterior condylar height.
